(19) United States
(12) Patent Application Publication
(10) Pub. No.: US 2023/0010375 A1
(43) Pub. Date: Jan. 12, 2023

(54) ANTI-VIRAL COMPOSITION

(71) Applicant: Noel Carroll, London (GB)

(72) Inventor: Noel Carroll, London (GB)

(21) Appl. No.: 17/947,314

(22) Filed: Sep. 19, 2022

Related U.S. Application Data
(63) Continuation of application No. PCT/IB2021/052288, filed on Mar. 18, 2021.

(30) Foreign Application Priority Data
Mar. 19, 2020 (GB) 2004022.6
Oct. 27, 2020 (GB) 2017046.0
Dec. 30, 2020 (GB) 2020747.8
Feb. 26, 2021 (GB) 2102819.6

(51) Int. Cl. A61K 31/19; A61P 31/14

(52) U.S. Cl. CPC A61K 31/19; A61P 31/14

(57) ABSTRACT

There is provided an active compound comprising bicarbonate of soda and/or magnesium carbonate for use in the treatment of a viral infection. As the pH of the host increases through administration of this pharmaceutical composition or preparation there may be a reduction in the ability of viral cells to fuse with the host cells. In any event, it has been found that administration of such a composition treats a viral infection or can act as a prophylaxis. The active compound may comprise only bicarbonate of soda (sodium bicarbonate), only magnesium carbonate or a mixture of bicarbonate of soda and magnesium carbonate.

### RELATED APPLICATIONS

This application claims the benefit under 35 U.S.C. § 120, and is a continuation, of co-pending International Application PCT/IB2021/052288, filed Mar. 18, 2021 and designating the US, which claims priority to GB Applications 2004022.6; 2017046.0; 2020747.8; and 2102819.6, filed Mar. 19, 2020; Oct. 27, 2020; Dec. 30, 2020; and Feb. 26, 2021, respectively, such GB Applications also being claimed priority to under 35 U.S.C. § 119. These GB and International applications are incorporated by reference herein in their entireties.

### FIELD

The present invention relates generally to the reduction or prevention of viral replication within a mammal, and finds particular, although not exclusive, utility in the reduction or prevention of enveloped viruses such as coronavirus and herpes.

### BACKGROUND

A viral infection in a host, such as a mammal, occurs when a virus infects a host through the introduction of the virus' genetic material into the cells of the host. Once introduced, the virus will replicate within the host's cells, releasing new viral particles within the host thereby spreading the virus further within the body of the host. This can lead to damage to the health of the host.

There are many anti-virals available which require the immune system of the host to help in targeting the virus. However, these generally require the infection to be under way before the host will mount a response and this will not always be successful if the host has a compromised immune system. Further, a host's immune system can go into overdrive in the presence of some viruses thereby causing even more damage to the host.

Research suggests that a virus, for example, an enveloped virus, has a reduced ability to fuse with its host and, therefore, replicate if the pH of the environment is altered towards an alkaline pH.

Viruses can exploit the pH of the host and often the replication of the virus will cause a lowering of the pH of the host. For example, virus introduced to a host may affect the functioning of the host's lungs, resulting in a reduced efficiency. Lowered lung efficiency prevents the lungs from expelling a sufficient amount of carbon dioxide, and consequently lowers the blood pH of the host.

### SUMMARY

In an embodiment of the present invention there is provided a composition comprising an active compound comprising bicarbonate of soda and/or magnesium carbonate for use in the treatment of a viral infection.

Bicarbonate of soda and magnesium carbonate are alkaline substances which may increase the pH of a host. As the pH of the host increases through administration of this pharmaceutical composition or preparation there may be, inter alia, a reduction in the ability of viral cells to fuse with the host cells. In any event, it has been found that administration of such a composition treats a viral infection or can act as a prophylaxis.

For avoidance of doubt the active compound may comprise only bicarbonate of soda (sodium bicarbonate), only magnesium carbonate or a mixture of bicarbonate of soda and magnesium carbonate.

### DETAILED DESCRIPTION

In the present context, “administration”, “administered” or derivatives thereof includes administration by a third party, for example a healthcare professional, or self-administration by the patient.

In the present context, the term “patient” includes those suffering from a disease and/or infection, those suffering from a disease and/or infection but taking prophylactic treatment for a different disease and/or infection and those not suffering from a disease and/or infection but taking prophylactic treatment.

Conveniently the composition is in liquid form. The liquid form can be for use in oral or parenteral administration. An oral preparation can be prepared by dissolving the composition in any suitable liquid, for example, water, orange juice, milk, tea or coffee. The preferable liquid is water. Additionally, the oral preparation may be in an edible jellied form or tablet form. Parenteral formulations can be formulated by those skilled in the art. Forms of parenteral administration comprise but are not limited to rectal, intravenous, topical and/or nasal forms. Different forms of administration can be used in the same administration period.

The pharmaceutical preparation may be provided to a user by dissolving the pharmaceutical preparation (composition) in a suitable amount of liquid such that the user may drink the solution. Volumes of liquid, such as water, which are contemplated are in the range of 250 to 2000 ml. Conveniently, the liquid is in the range of 750 to 1500 ml.

Conveniently the composition is provided in a form which would allow at least a temporary coating of the nasal passageways and/or esophagus thereby raising the surface pH of cells therein.

The pharmaceutical composition may be mixed with any suitable flavor enhancer, emulsifier, surfactant, stabilizer, flavoring or excipient. Such compounds will be well known to those in the art. For example, it may be necessary to improve the taste of the composition. Citric acid can be used in the composition.

Conveniently the virus is selected from coronavirus, rhinovirus, RSV, influenza virus or herpes virus.

Conveniently the viral infection is caused by an enveloped virus. The fusion of an enveloped virus with a host cell will be altered in alkaline conditions. Conveniently, the enveloped virus is selected from coronavirus, RSV, influenza virus or herpes virus. Conveniently, the virus is coronavirus and/or herpes virus. Herpes virus such as varicella-zoster can be a complication in coronavirus cases. The herpes virus is a latent virus which can opportunistically exploit an immune system which is compromised or otherwise overloaded. As an enveloped virus it has a similar mechanism of action to coronavirus and, therefore, can be targeted by the present composition. Conveniently, the virus is coronavirus.

In an embodiment of the invention the composition of the present invention is for use in a method of treatment of a viral infection characterized in that a single dose amount C of active compound is provided in grams and calculated by dividing a recipient's weight A, in kilograms, by 10 to provide B and multiplying B by 6 wherein the single dose amount C present in the composition can be in the range of C−30% to C+40%.

Conveniently the single dose amount C is in the range of C−20% to C+30%

Conveniently the single dose amount of C is C.

Conveniently the recipient's weight A kg is in the range of 25 kg and 80 kg. The composition of the present invention can be used for adults and children over 9 years old. Recipients below 25 kg and children of a younger age may also take the composition with or without supervision.

A single dose may be taken to significantly increase the pH of the patient thereby disrupting the viral infection or potential viral infection.

As mentioned previously, the active compound may be only bicarbonate of soda, only magnesium carbonate or a mixture of bicarbonate of soda and magnesium carbonate. In the mixture the ratio of bicarbonate of soda to magnesium carbonate is grams:grams and may be any ratio convenient to those skilled in the art, for example from 10:90 to 90:10.

Conveniently the ratio is between 30:70 to 70:30 or between 40:60 to 60:40, or 50:50.

Conveniently a single dose is divided into a partial dose and is administered at least twice per administration period.

Conveniently the partial dose is administered two to five times per administration period.

Conveniently the partial dose is administered two to three times per administration period.

Conveniently the partial doses are equal amounts of active compound. If the doses are of equal amount, then the alkalinity of the patient may be maintained at a steady rate over the administration period.

Conveniently the partial doses are not equal amounts of active compound. If the doses are not of an equal amount, then the regime can be altered to provide a significant raising of the pH of a patient at a certain time or for a certain period. This can be monitored by any suitable method by a person skilled in the art. It would also allow for, for example, a larger dose to be given before bedtime in order that the raised pH was maintained during the night.

Conveniently the administration period is between substantially 1 to 6 days. In the present context, “1 day” can mean any time period within 24 hours. For example, substantially 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, or 24 hrs.

Conveniently the administration period is between substantially, 1 to 5 days or 2 to 5 days.

Conveniently the administration period is between substantially 2 to 3 days. If symptoms persist or a viral test post-administration is positive after the single dose is taken, then further single doses can be administered.

Conveniently partial doses are administered at intervals during the administration period.

Conveniently the intervals are substantially equal. Alternatively, the intervals can be altered such that they are not equal in order that, for example, the patient does not need to take a dose during a rest period. If the partial doses are administered at least once per 12 to 16-hour period, then the partial doses can be administered during waking hours.

If the partial doses are administered regularly and/or equally throughout the administration period, then the viral numbers in the host may be kept to a minimum thereby increasing the likelihood that the composition will be efficacious.

If the partial doses are administered at inequal intervals then this may result in a raising of the pH to allow for protection during, for example, periods of sleep. In other words, for example, two partial doses could be given close to each other around bedtime.

If the viral numbers can be kept at a low level in the host for a sustained period by the composition, then the immune system of the host may prevent the virus overwhelming the host. Accordingly, the number and/or frequency of partial doses administered may be dependent upon the health of the host pre-infection.

Taking the pharmaceutical preparation may cause the pH level of a host of a virus to increase despite the reduction of pH levels caused by a decreased lung efficiency triggered by the virus and/or the virus replication mechanism.

The increase in the host's pH level may reduce or prevent the ability of the virus within the host to replicate, thereby reducing the damage to the health of the host.

The ingestion of the active compound can help the human body destroy the virus.

In an embodiment of the present invention there is provided a method of prophylaxis or treatment of a viral infection, the method comprising administering partial doses of the composition according to the present invention to a patient over an administration period. If symptoms persist or a viral test post-administration is positive, then after the single dose is taken then further single doses can be administered.

Conveniently, the pH of the urine of the patient is regularly monitored and the partial doses are administered to maintain the pH of the urine above 7. The pH of the urine can be monitored by any suitable method known to a person skilled in the art.

Conveniently, the pH of the urine is maintained above 7 for at least 8 hours. Conveniently, the pH of the urine is maintained above 7 for up to 72 hours.

Conveniently the pH of the urine is maintained above 7.5.

Embodiments of the present invention will now be described by way of example.

### Example 1

Patient A had a positive COVID-19 test. Patient A weighed 80 kg. On Day 1 after the positive test, Patient A drank a part dose of 16 grams or ⅓rd of a dose of bicarbonate of soda dissolved in 250 ml of water. They followed up the first part dose with a second part dose of 16 grams being another ⅓rd of a dose of bicarbonate of soda dissolved in 250 ml of water within 4 hours of the first. Then the last part of the dose of 16 grams of bicarbonate of soda being another ⅓rd of a dose (total 3/3rds) within another 4 to 24 hours.

The patient often presents with no symptoms 8 hours after the first ⅔rds of a dose. However, in order to prevent symptoms returning, the 3rd dose must be administered.

A second swab test was taken by Patient A 1 day after the last part dose. It has been found that this swab test may prove a non-conclusive test. The high levels of sodium bicarbonate tend to confuse this second Covid-19 test. However, if a third Swab test is undertaken 2 days after the second Swab test, if it proved non-conclusive, it has been found that the third Swab test should then prove negative to Covid-19.

If the test is not negative the composition should be administered again.

### Example 2

If Patient B had a positive COVID-19 test and weighed 72 kg, Patient B should be administered 43.2 g of active compound. The active compound was prepared by mixing 21.6 g of bicarbonate of soda and 21.6 g of magnesium carbonate. On Day 1 after the positive test, Patient B would drink a part dose of 21.6 g grams or ½ of a dose of active compound dissolved in 400 ml of water. They would then follow up the first part dose with a second part dose of 10.8 grams being another ¼ of a dose of active compound dissolved in 250 ml of water within 2 hours of the first. Then the last part of the dose of 10.8 grams of active compound being another ¼ of a dose within another 2 to 24 hours.

The patient often presents with no symptoms 8 hours after the first two doses. However, in order to prevent symptoms returning, the third dose must be administered.

A second swab test would be taken by Patient B 1 day after the last part dose. It has been found that this swab test may prove a non-conclusive test. The high levels of active compound may confuse this second Covid-19 test. However, if a third swab test is undertaken 2 days after the second swab test, if it proved non-conclusive, it has been found that the third swab test should then prove negative to Covid-19.

If the test is not negative the composition should be administered again.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof. Thus, the scope of the expression “a device comprising means A and B” should not be limited to devices consisting only of components A and B. It means that with respect to the present invention, the only relevant components of the device are A and B.

Reference throughout this specification to “an embodiment” or “an aspect” means that a particular feature, structure or characteristic described in connection with the embodiment or aspect is included in at least one embodiment or aspect of the present invention. Thus, appearances of the phrases “in one embodiment”, “in an embodiment”, or “in an aspect” in various places throughout this specification are not necessarily all referring to the same embodiment or aspect, but may refer to different embodiments or aspects. Furthermore, the particular features, structures or characteristics of any one embodiment or aspect of the invention may be combined in any suitable manner with any other particular feature, structure or characteristic of another embodiment or aspect of the invention, as would be apparent to one of ordinary skill in the art from this disclosure, in one or more embodiments or aspects.

Similarly, it should be appreciated that in the description various features of the invention are sometimes grouped together in a single embodiment, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Moreover, the description of any individual drawing or aspect should not necessarily be considered to be an embodiment of the invention. Rather, as the following claims reflect, inventive aspects lie in fewer than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some features included in other embodiments, combinations of features of different embodiments are meant to be within the scope of the invention, and form yet further embodiments, as will be understood by those skilled in the art. For example, in the following claims, any of the claimed embodiments can be used in any combination.

In the description provided herein, numerous specific details are set forth. However, it is understood that embodiments of the invention may be practiced without these specific details. In other instances, well-known methods, structures and techniques have not been shown in detail in order not to obscure an understanding of this description.

In the discussion of the invention, unless stated to the contrary, the disclosure of alternative values for the upper or lower limit of the permitted range of a parameter, coupled with an indication that one of said values is more highly preferred than the other, is to be construed as an implied statement that each intermediate value of said parameter, lying between the more preferred and the less preferred of said alternatives, is itself preferred to said less preferred value and also to each value lying between said less preferred value and said intermediate value.

The use of the term “at least one” may mean only one in certain circumstances. The use of the term “any” may mean “all” and/or “each” in certain circumstances.

The principles of the invention will now be described by a detailed description of at least one drawing relating to exemplary features. It is clear that other arrangements can be configured according to the knowledge of persons skilled in the art without departing from the underlying concept or technical teaching, the invention being limited only by the terms of the appended claims.

1. A method of prophylaxis or treatment of a viral infection, the method comprising:
detecting urine pH of a patient;
administering bicarbonate of soda and/or magnesium carbonate to the patient during an administration period;
repeating the detecting during the administration period; and
repeating the administering during the administration period if the detecting determines the administering is necessary to maintain the urine pH above 7 during the administration period.

2. The method of claim 1, wherein the viral infection is caused by at least one of a coronavirus, rhinovirus, RSV, influenza virus, and herpes virus.

3. The method of claim 1, wherein the administration period is 1 to 6 days.

4. The method of claim 3, wherein the administration period is 2 to 5 days.

5. The method of claim 1, wherein the administering administers the bicarbonate of soda and/or magnesium carbonate in a range of 0.7-1.4 grams per kilogram weight of the patient multiplied by 0.6.

6. The method of claim 5, wherein the range is 0.8-1.3 grams per kilogram weight of the patient multiplied by 0.6.

7. The method of claim 6, wherein the range is 1 gram per kilogram weight of the patient multiplied by 0.6.

8. The method of claim 5, wherein the weight of the patient is 25-80 kilograms.

9. The method of claim 1, wherein the administering is performed at least twice, and wherein all the administerings combined administer the bicarbonate of soda and/or magnesium carbonate in a range of 0.7-1.4 grams per kilogram weight of the patient multiplied by 0.6.

10. The method of claim 9, wherein the administering is performed 2 or 3 times.

11. The method of claim 9, where each of the administerings administer equal amounts of the bicarbonate of soda and/or magnesium carbonate.

12. The method of claim 9, where each of the administerings administer unequal amounts of the bicarbonate of soda and/or magnesium carbonate.

13. The method of claim 1, wherein the bicarbonate of soda and/or magnesium carbonate is administered in a liquid dosage.

14. The method of claim 1, wherein the administration period is at least 8 hours in which the urine pH is maintained above 7.

15. The method of claim 14, wherein the administration period is at least 72 hours in which the urine pH is maintained above 7.

16. The method of claim 1, wherein the repeatings maintain the urine pH above 7.5 during the administration period.
